(19) United States
(12) Patent Application Publication
(10) Pub. No.: US 2024/0058050 A1
(43) Pub. Date: Feb. 22, 2024

(54) MEDICAL DEVICES AND RELATED METHODS FOR DELIVERING ELECTRICAL ENERGY AND/OR INJECTING FLUID

(71) Applicant: Boston Scientific Scimed, Inc., Maple Grove, MN (US)

(72) Inventors: Todd Mannion PFIZENMAIER, Sturbridge, MA (US); Gary A. JORDAN, Litchfield, NH (US); Anthony Frank TASSONI, Andover, MN (US)

(73) Assignee: Boston Scientific Scimed, Inc., Maple Grove, MN (US)

(21) Appl. No.: 18/450,007

(22) Filed: Aug. 15, 2023

Related U.S. Application Data
(60) Provisional application No. 63/398,584, filed on Aug. 17, 2022.

(51) Int. Cl. A61B 18/14

(52) U.S. Cl. CPC A61B 18/14; A61B 2018/1412; A61B 2018/00601

(57) ABSTRACT

A medical device includes a handle, including a movable body. The medical device also includes a shaft extending from the handle, and an electrode at a distal end of the shaft. Movement of the movable portion of the handle controls a position of the electrode relative to the distal end of the shaft. At least a portion of the shaft includes a braiding.

### CROSS-REFERENCE TO RELATED APPLICATIONS

This patent application claims the benefit of priority under 35 U.S.C. § 119 to U.S. Provisional Patent Application No. 63/398,584, filed Aug. 17, 2022, which is herein incorporated by reference in its entirety.

### TECHNICAL FIELD

Aspects of this disclosure generally relate to medical devices and related methods. In particular, aspects of this disclosure relate to medical devices and related methods configured for the treatment of tissue by delivering electrical energy to or into tissue, via an electrode, and/or injecting fluid into and/or under tissue.

### BACKGROUND

Medical devices, such as endoscopes or other suitable insertion devices, are employed for a variety of types of diagnostic and surgical procedures, such as endoscopy, laparoscopy, arthroscopy, gynoscopy, thoracoscopy, cystoscopy, etc. Many of these procedures involve delivering energy to tissue of an organ or a gland to treat tumors, infections, and the like. Examples of such procedures include Endoscopic Mucosal Resection (EMR), Endoscopic Sub-mucosal Resection (ESR), Endoscopic Sub-mucosal Dissection (ESD), polypectomy, mucosectomy, etc. In particular, such procedures may be carried out by inserting an insertion device into a subject's body through a surgical incision, or via a natural anatomical orifice (e.g., mouth, vagina, or rectum), and performing the procedure or operation at a target site with an auxiliary device inserted through the insertion device.

At times, during a medical procedure, a user may use an injection needle and/or an energy delivery device for purposes of raising, separating, flushing, cutting, dissecting, ablating, marking, coagulating, cauterizing, or otherwise treating and/or manipulating tissue. The injection needle and/or the energy delivery device may need to traverse a tortuous path or anatomy while being maneuvered or otherwise delivered to the treatment site and/or to the tissue being treated.

The devices and methods of this disclosure may rectify one or more of the deficiencies described above or address other aspects of the art.

### SUMMARY

Examples of this disclosure relate to, among other things, medical devices configured for treating tissue by delivering electrical energy to the tissue, and configured for delivering fluid into and/or under the tissue. Each of the examples disclosed herein may include one or more of the features described in connection with any of the other disclosed examples.

In some aspects, a medical device may include a handle, a shaft extending from the handle, and an electrode at a distal end of the shaft. The handle may include a movable body. Movement of the movable body of the handle may control a position of the electrode relative to the distal end of the shaft. At least a portion of the shaft may include a braiding.

The medical device may include one or more of the following features. The braiding may be a polymer. The medical device may be an electrosurgical knife. The shaft may further include an outer coating. The braiding may be embedded within the outer coating. The braiding may include a plurality of threads over at least a portion of the shaft. The medical device may include an inner shaft. The plurality of threads may be deposited over at least a portion of the inner shaft. The braiding may extend along an entire length of the inner shaft. The outer coating may be formed of polyether ether ketone (PEEK).

The handle may include a port and an electrical connection. The handle and the shaft may connect the port and the electrical connection to the electrode. Movement of the movable body of the handle may control a position of the electrode relative to the distal end of the shaft without delay. The braiding may be configured to minimize kinking of the shaft or a drive element connecting the movable body to the electrode.

A distal portion of the shaft may further include a ring radially surrounding a portion of the braiding. The ring may be a radiopaque marker band. The medical device may further include an end cap coupled to the distal end of the shaft. A portion of the end cap may extend distally of the distal end of the shaft to form an exposed portion of the end cap. The end cap may be at least partially insulative. The ring may be positioned proximal of the exposed portion of the end cap. The handle may include one or more indicators. The one or more indicators may be indicative of at one or more configurations of the electrode relative to the distal end of the shaft. The one or more indicators may include a first indicator and a second indicator. The first indicator may be indicative of a retracted configuration of the electrode. The second indicator may be indicative of an extended configuration of the electrode.

In another aspect, a medical device may include a handle, a shaft extending from the handle, and an electrosurgical knife at a distal end of the shaft. At least a portion of the shaft may include a braiding that includes a plurality of polymer threads.

The medical device may include one or more of the following features. The handle may include a movable body. Movement of the movable body may control a position of the electrosurgical knife relative to the distal end of the shaft. The medical device may include an inner shaft. The plurality of polymer threads may be deposited over at least a portion of the inner shaft. The shaft may further include an outer coating formed of polyether ether ketone (PEEK). The braiding may be embedded within the outer coating.

In yet another aspect, a medical device may include a handle, a shaft extending from the handle, and an electrode at a distal end of the shaft. The handle may include a movable body. Movement of the movable body may control a position of the electrode relative to the distal end of the shaft. At least a portion of the shaft may include a braiding that includes a plurality of threads over at least a portion of the shaft.

The medical device may include one or more of the following features. The medical device may include an inner shaft. The plurality of threads may be deposited over at least a portion of the inner shaft. The shaft may further include an outer coating. The braiding may be embedded within the outer coating.

It may be understood that both the foregoing general description and the following detailed description are exemplary and explanatory only and are not restrictive of the disclosure, as claimed.

### BRIEF DESCRIPTION OF THE DRAWINGS

The accompanying drawings, which are incorporated in and constitute a part of this specification, illustrate exemplary aspects of the present disclosure and together with the description, serve to explain the principles of the disclosure.

FIG. 1 illustrates an exemplary medical device, according to aspects of this disclosure.

FIG. 2 illustrates a distal portion of the exemplary medical device of FIG. 1, according to aspects of this disclosure.

FIG. 3 is a longitudinal cross-sectional view of a portion of the distal portion of the exemplary medical device of FIG. 1, according to aspects of this disclosure.

### DETAILED DESCRIPTION

Examples of this disclosure include devices and methods for: facilitating and improving the efficacy, efficiency, and safety of treating and/or manipulating tissue when, for example, applying electrical energy to tissue with an electrode; delivering fluid into and/or under tissue during a medical procedure through or around the distal end of the electrode; and insulating a distal tip of the electrode. For example, aspects of this disclosure may provide a user (e.g., physician, medical technician, or other medical service provider) with the ability to apply electrical energy or heat to tissue using a medical device having an electrode, and to deliver fluid into and/or under tissue with the same medical device. Aspects of this disclosure may provide the user with the ability to apply electrical energy or heat and deliver fluid with a reduced likelihood of damaging tissue or contacting unintended portions of the tissue. Some aspects of this disclosure may be used in performing an endoscopic, laparoscopic, arthroscopic, gynoscopic, thoracoscopic, cystoscopic, or other type of procedure. Additionally, some aspects of this disclosure may help manipulate or otherwise deliver the medical device to a treatment site.

Reference will now be made in detail to examples of this disclosure described above and illustrated in the accompanying drawings. Wherever possible, the same reference numbers will be used throughout the drawings to refer to the same or like parts.

The terms “proximal” and “distal” are used herein to refer to the relative positions of the components of an exemplary medical device. When used herein, “proximal” refers to a position relatively closer to the exterior of the body of a subject or closer to a user, such as a medical professional, holding or otherwise using the medical device. In contrast, “distal” refers to a position relatively further away from the medical professional or other user holding or otherwise using the medical device, or closer to the interior of the subject's body. As used herein, the terms “comprises,” “comprising,” “has,” “having,” “includes,” “including,” or other variations thereof, are intended to cover a non-exclusive inclusion, such that a device or method that comprises a list of elements does not include only those elements, but may include other elements not expressly listed or inherent thereto. Unless stated otherwise, the term “exemplary” is used in the sense of “example” rather than “ideal.” As used herein, the terms “about,” “substantially,” and “approximately,” indicate a range of values within +/−10% of a stated value.

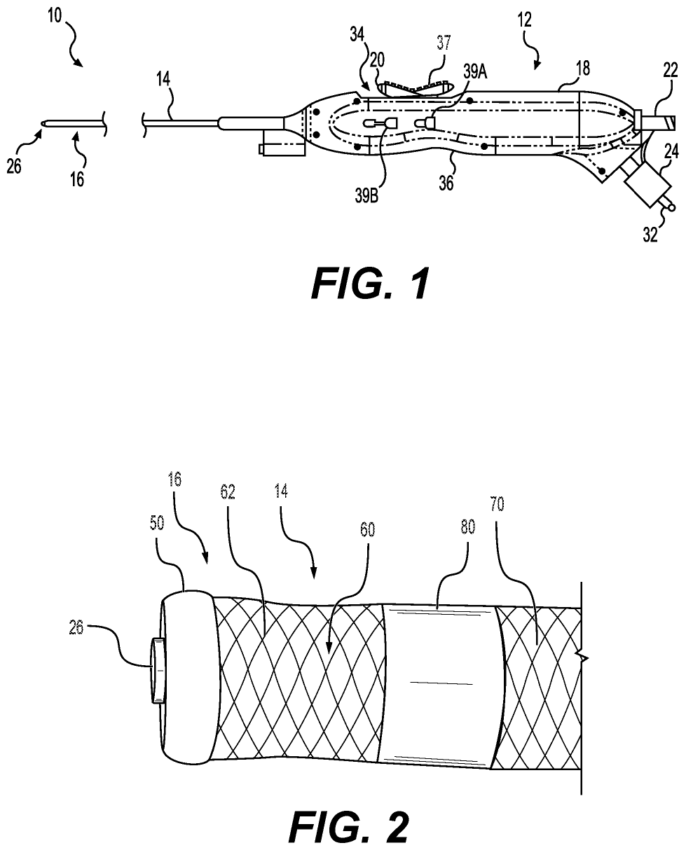

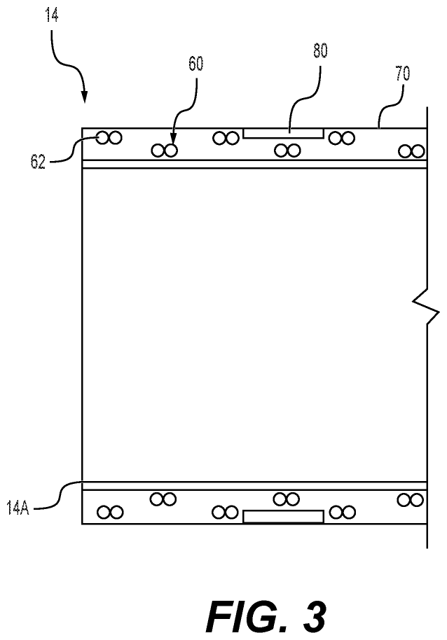

FIG. 1 depicts a medical device 10 that includes a handle 12, a shaft 14, and a distal end 16. Handle 12 may include a main body 18 and a movable body 20. Handle 12 also may include a port 22 configured to receive fluid, and a hub 24 configured to receive electrical energy, similar to an electrical plug or socket. Distal end 16 includes an end effector, for example, an electrode portion 26 (hereinafter “electrode 26”). Electrode 26 is electrically connected to hub 24, and as discussed in detail below, may include a channel fluidly connected to, or otherwise in fluid communication with, port 22. In these aspects, electrode 26 may form an electrosurgical knife. Additionally, electrode 26 may include one or more depictions or indicators 39A and 39B on handle 12, and indicators 39A and 39B may be indicative of a position of electrode 26. For example, electrode 26 may be positioned in at least a first or proximally retracted configuration relative to distal end 16 (e.g., as shown in indicator 39A) and a second or distally extended configuration relative to distal end 16 (i.e., as shown in indicator 39B). Electrode 26 may be movable between the at least first and second configurations (corresponding to indicators 39A and 39B) via movement of movable body 20. Moreover, as discussed below, one or more portions of shaft 14 may include a braiding 60 (FIGS. 2 and 3). For example, in at least some aspects, an entirety of shaft 14 may include braiding 60, such that braiding 60 extends along an entire length of shaft 14.

Medical device 10 may be inserted into a body lumen of a subject, either through an insertion device (not shown) or alone, such that at least a portion of shaft 14 may be within the subject, while handle 12 may remain outside of the subject. Distal end 16 may be positioned at a target site within the subject. For example, distal end 16 may traverse a tortuous path or anatomy while being maneuvered or otherwise delivered to the treatment site and/or to the tissue being treated. From outside of the subject, a user can manipulate handle 12. Movement of movable body 20 relative to main body 18 in a first direction (e.g., the distal direction) may extend electrode 26 relative to shaft 14 (e.g., move electrode 26 distally relative to a distal end of shaft 14). For example, movement of movable body 20 relative to main body 18 in the first or distal direction may transition electrode 26 from a retracted configuration (e.g., the configuration shown by indicator 39A) to an extended configuration (e.g., the configuration shown by indicator 39B). Similarly, movement of movable body 20 relative to main body 18 in a second direction (e.g., the proximal direction) may retract electrode 26 relative to shaft 14 (e.g., move electrode 26 proximally relative to a distal end of shaft 14). For example, movement of movable body 20 relative to main body 18 in the second or proximal direction may transition electrode 26 from an extended configuration (e.g., the configuration shown by indicator 39B) to a retracted configuration (e.g., the configuration shown by indicator 39A). Although not shown, movable body 20 or additional components of handle 12 may articulate electrode 26 and/or distal end 16 left or right, and/or up or down, relative to shaft 14.

Handle 12 may be coupled to a fluid source (not shown) via port 22. Port 22 may be in fluid communication with electrode 26 via an internal lumen (not shown), which may extend through handle 12 (FIG. 1) and shaft 14. It is noted that various portions of handle 12 shown in FIG. 1 may not be to scale, in order to more fully illustrate various portions of handle 12. In one aspect, the internal lumen may extend longitudinally through main body 18 of handle 12 and shaft 14 to fluidly connect port 22 to electrode 26. Port 22 may be positioned on a proximal portion of main body 18, for example, a proximal end of main body 18. Alternatively, port 22 may be positioned on a distal or central portion of main body 18. Moreover, port 22 may include a one-way valve, a luer, a seal, threading, and/or any appropriate element to help maintain a secure connection between handle 12 (and particularly port 22) and the fluid source, minimize or prevent back-flow (e.g., fluid flowing proximally out of port 22), and/or minimize or prevent leakage. In one example, a one-way valve may include an outer housing containing an inner elastomeric and/or gelatinous sealing member (not shown).

Handle 12 may be coupled to an energy source (not shown) via hub 24. Hub 24 may include one or more prongs or pins 32 to couple to the energy source. Hub 24 may be electrically coupled to electrode 26 via a conductive element (not shown), which may be electrically coupled to pin 32 and extend through handle 12 and through at least a portion of shaft 14. The energy source may be an electrocautery source, a radio frequency generator, a heating source, a current generator, etc. In one aspect, medical device 10 may be used for monopolar electrosurgery, and may include a return electrode positioned remotely from electrode 26 on or otherwise adjacent the subject. In another aspect, medical device 10 may be used for bipolar electrosurgery. In that instance, electrode 26 may include an active electrode portion, and a return electrode may be provided at or near another portion of electrode 26 and/or shaft 14. In one example, although not shown, two conductive elements may extend through shaft 14, where the conductive elements may be electrically isolated from each other, allowing one to conduct energy to the active electrode and the other to conduct energy from a return electrode.

Hub 24 may be positioned on main body 18, for example, on a proximal end of main body 18. In one aspect, port 22 may extend from the proximal end of main body 18 in a direction parallel to a longitudinal axis of main body 18, and hub 24 may extend from the proximal end of main body 18 at an angle transverse (e.g., approximately 45 degrees) to the longitudinal axis of main body 18. In another aspect, hub 24 may be positioned on a distal or central portion of main body 18, or on movable body 20. Although not shown, main body 18 and/or hub 24 may include a one-way valve, a luer, a seal, threading, and/or any appropriate element to help maintain a secure connection between handle 12 and the energy source, minimize or prevent back-flow (e.g., fluid flowing from port 22 and/or the internal lumen and proximally out of hub 24), and/or minimize or prevent leakage. Furthermore, although not shown, pin 32 may extend through hub 24 transverse to a longitudinal axis of handle 12, and may be electrically and physically connected to a conductive element (not shown), such as a wire, a cable, etc. The conductive element may extend longitudinally through the internal lumen in handle 12 and through a lumen in shaft 14, or otherwise through shaft 14. In one aspect, the conductive element may include one or more layers of insulation to help insulate the conductive element from the fluid in the internal lumen, for example, as an insulated wire. As described above, a second conductive element (not shown) may be provided as a return pathway where medical device 10 has a bipolar configuration. Although not shown, in another aspect, the energy source may be a part of handle 12 (e.g., an internal battery in handle 12).

As mentioned, handle 12 may control the extension or retraction of electrode 26 relative to the distal end 16 of shaft 14. For example, main body 18 may include a slot 34, and movable body 20 may be slidably positioned within slot 34. For example, main body 18 may be configured to be held by a user's hand, and movable body 20 may be configured to be controlled by the movement of the user's thumb. For example, a side of main body 18 opposite to movable body 20 may include one or more contours 36, which may help the user grip main body 18. Additionally, movable body 20 may include one or more ridges 37, which may help the user manipulate movable body 20. Movable body 20 may be lockable in one or more positions relative to main body 18, and/or may be spring-biased in a direction (e.g., toward a proximally retracted position).

Movable body 20 may be coupled to a drive element (not shown), and the drive element may impart distal or proximal movement to at least a portion of electrode 26 based on relative movement between main body 18 and movable body 20. In one aspect, the conductive element (not shown) may also act as a drive wire, rod, cable, or the like. In this aspect, the conductive element imparts distal or proximal movement to at least a portion of electrode 26 while also coupling electrode 26 to hub 24, e.g., the one or more pins 32, to deliver the energy to (and/or from) electrode 26.

As shown in FIG. 1, handle 12 may also include one or more indicators, for example, indicators 39A, 39B. For example, indicators 39A, 39B may visually indicate to the user the position of electrode 26 relative to shaft 14. The position of indicators 39A, 39B may also correspond with the position of movable body 20. For example, indicator 39A may be positioned on handle 12 at a position corresponding with a retracted position of movable body 20, and may indicate that electrode 26 is retracted relative to shaft 14. Similarly, indicator 39B may be positioned on handle 12 at a position corresponding with an extended position of movable body 20, and may indicate that electrode 26 is extended relative to shaft 14.

As shown in FIG. 1, shaft 14 extends from a distal portion of main body 18 to distal end 16, and may surround at least a portion of electrode 26. Shaft 14 may be a sheath that surrounds at least a portion of one or more lumens and the drive element (not shown). In another aspect, shaft 14 may be an extrusion that includes one or more lumens extending from handle 12 to distal end 16. The lumens may receive a proximal portion of electrode 26, the drive element, the conductive element, and any other elements of device 10 that are discussed above and extend between handle 12 and distal end 16.

FIG. 2 illustrates a distal portion of shaft 14, including distal end 16. As shown in FIG. 2, shaft 14 may include or otherwise be coupled to an end cap 50. End cap 50 may be at least partially insulating. For example, end cap 50 may be formed of a ceramic (e.g., zirconia, an alloy containing zirconium (e.g., ZrO₂), aluminum oxide (Al₂O₃), a ceramic alloy, etc.), a polymer material (e.g., a fluoropolymer, polyether ether ketone (PEEK), etc.), or another medically-safe, heat-resistant, and non-conductive material.

Furthermore, a distal portion of electrode 26 may be larger (i.e., wider) than the lumen within shaft 14 and/or end cap 50, for example, such that the distal portion of electrode 26 remains distal of shaft 14 and/or end cap 50 when electrode 26 is fully retracted proximally, as shown in FIG. 2. One or more portions of electrode 26 may be formed of a conductive material, for example, a stainless steel (e.g., 316L stainless steel), titanium, or another medically-safe and conductive material. In one aspect, electrode 26 may include a surface finish, for example, may be passivated per ASTM A967 Nitric 2.

Moreover, as shown in FIG. 2, shaft 14 includes braiding 60. Braiding 60 includes a plurality of threads 62, for example, woven over one or more portions of shaft 14. Threads 62 may be formed of a plastic material (e.g., a polymeric material) or a non-metallic material. Alternatively, threads 62 may be formed of a metallic material. In some aspects, braiding 60 may include approximately 12 to approximately 40 threads 62, for example, approximately 16 or 32 threads 62. In some aspects, threads 62 may include one wire or strand, or a plurality of wires or strands (e.g., two, three, four, or more wires or strands) wound, twisted, adjacent, or otherwise coupled or positioned together. As discussed in detail below, braiding 60 and its threads 62 may be an intermediate layer in shaft 14.

Threads 62 may include a constant or consistent spacing and/or braid angle over at least a distal portion of shaft 14. In some aspects, threads 62 include a constant or consistent spacing and/or braid angle over an entirety of the length of shaft 14. Moreover, threads 62 may terminate proximal of end cap 50. End cap 50 may help to insulate threads 62 (e.g., if threads 62 are metallic). In some aspects, threads 62 may be applied to one or more portions (e.g., an entirety) of shaft 14 with a braider machine. Each thread 62 may have a size (i.e., a cross-sectional size) of approximately 0.0005 inches to approximately 0.005 inches. Each thread 62 may include a circular cross-section, including a circular cross-sectional diameter of approximately 0.0005 inches to approximately 0.005 inches. Alternatively, one or more of threads 62 may include a “D” shaped cross-sectional shape, an oval or ovular cross-sectional shape, or another non-circular cross-sectional shape. In some examples, threads 62 may each include a thickness (e.g., a cross-sectional diameter) between approximately 0.001 inches to approximately 0.003 inches, for example, approximately 0.002 inches. In some aspects, the thickness of threads 62 may help to optimize and/or balance the flexibility and wall thickness of shaft 14 with braiding 60. Moreover, in some aspects, braiding 60 may include a pick spacing over braiding 60 (i.e., picks per inch or p.p.i.) of approximately 70 p.p.i. to approximately 110 p.p.i., for example, approximately 90 p.p.i. In these aspects, the picks per inch of braiding 60 on shaft 14 may help to control the stiffness and/or flexibility of shaft 14.

Shaft 14 may include an outer coating 70 (e.g., a thermoplastic polymer coating). For example, outer coating 70 may include or be formed of a polyether ether ketone (PEEK), Pebax, Hyrel, Arnitel, Polyurethane (PU), Polytetrafluoroethylene (PTFE), and may at least partially radially surround braiding 60. For example, braiding 60 may be completely embedded within coating 70. In some aspects, each thread 62 of braiding 60 may be entirely radially surrounded by coating 70. In other aspects, a portion (e.g., outer and/or side portion(s)) of each thread 62 of braiding 60 may be covered by coating 70. In any of these aspects, outer coating 70 may include a high dielectric strength and/or be formed of or include one or more flouropolymers. If threads 62 are formed of a metallic material, outer coating 70 may help to insulate threads 62, for example, to help ensure that energy delivered to the treatment site by electrode 26 is not conveyed to threads 62. As mentioned above, threads 62 may terminate proximal of end cap 50. In this aspect, outer coating 70 may at least partially encapsulate distal ends of threads 62, for example, to help insulate threads 62 (e.g., if threads 62 are formed of metallic material) or otherwise encapsulate threads 62, such that threads 62 do not interfere with the delivery, movement, or other manipulation of shaft 14 and/or electrode 26, or the treatment of tissue at the treatment site.

In some aspects, shaft 14 may include an extrusion. For example, the extrusion may form an inner portion of shaft 14. The extrusion may include one or more working channels or lumens, as discussed above. Braiding 60 may be wound around the extrusion (e.g., by the braiding machine). Then, outer coating 70 may be extruded, deposited, formed, or otherwise positioned radially outside of braiding 60. Coating 70 may flow around and within braiding 60, filling the openings between threads 62, to encapsulate braiding 60.

Furthermore, in some aspects, a distal portion of shaft 14 may include a ring 80, for example, a mark ring, such as a radiopaque marker band. Ring 80 may be positioned radially around a distal portion of shaft 14, for example, proximal of an exposed portion of end cap 50. In some aspects, ring 80 may be positioned radially around or outside of coating 70. In other aspects, at least a portion of ring 80 may be covered or encapsulated by coating 70. Ring 80 may help the user to visualize a position of shaft 14 and/or electrode 26, for example, relative to a distal end of an insertion device (not shown). Alternatively or additionally, a distal portion of shaft 14 may include one or more other non-radiopaque visual indicators, for example, one or more etched (e.g., laser etched) visual indicators. For example, shaft 14 may include one or more non-radiopaque visual indicators, which may be formed using a Keyence ultraviolet (UV) laser mark or etch. In some aspects, the insertion device may include an internal lumen to receive shaft 14, and a distal end of the insertion device may include a visualization device (e.g., a camera) and/or an illumination device (e.g., one or more light emitting diodes). The visualization device and/or the illumination device may help the user to visualize the extension, movement, position, manipulation, etc. of shaft 14 and/or electrode 26. In these aspects, ring 80 may help the user to determine a position of shaft 14 and/or electrode 26 relative to the insertion device and/or the visualization device. Ring 80 may, for example, help the user to ensure that shaft 14 and/or electrode 26 are positioned a safe distance and/or position relative to the insertion device and/or the visualization device at the treatment site.

FIG. 3 illustrates a longitudinal cross-sectional view of a portion of shaft 14, for example, a distal portion of shaft 14. It is noted, however, that internal elements of medical device 10, for example, including electrode 26 and/or other mechanical and/or fluidic connections, are omitted in FIG. 3. Nevertheless, as shown, a portion of device 10 (e.g., shaft 14) may include an inner layer or shaft 14A, for example, formed via the extrusion. In some aspects, inner shaft 14A may be formed of a polymeric and/or fluoropolymeric material, for example, Polytetrafluoroethylene (PTFE), polyether ether ketone (PEEK), etc. Additionally, a plurality of threads 62 may be braided, deposited, formed, or otherwise positioned on the radial exterior of at least a portion of inner shaft 14A, for example, to form braiding 60. As mentioned, each of threads 62 may include one or more wires or strands. In some aspects, two or more threads 62 may be deposited, formed, or otherwise positioned on inner shaft 14A adjacent to one another. Furthermore, as shown in FIG. 2, threads 62 may also alternate being above or below other threads (e.g., woven) to form braiding 60. In some aspects, threads 62 may be braided in a “one over, one under” pattern. In other aspects, threads 62 may be braided in a “two over, two under” pattern. The braiding pattern may help to affect mechanical properties of braiding 60 and/or shaft 14. Moreover, in some aspects, one or more portions of shaft 14 (e.g., inner shaft 14A) may be formed with or otherwise include a hydrophilic coating. For example, one or more inner portions of shaft 14 (e.g., an inner diameter of inner shaft 14A) and/or one or more outer portions of shaft 14 (e.g., an outer diameter of inner shaft 14A) may include a hydrophilic coating.

Outer coating 70 may then be extruded, deposited, formed, or otherwise positioned on threads 62 and the radially exterior of inner shaft 14A. In some aspects, outer coating 70 may be deposited, formed, or otherwise positioned surrounding one or more threads 62, for example, radially between inner shaft 14A and threads 62 and also radially outside of threads 62. Outer coating 70 may also be deposited, formed, or otherwise positioned between threads 62, filling the spaces/openings between adjacent threads 62. As mentioned above, ring 80 may also be positioned radially outside of outer coating 70, or alternatively, may be at least partially encapsulated by outer coating 70. In these aspects, ring 80 may be formed of a non-conductive and/or non-metallic material. Furthermore, in some aspects, shaft 14 may include one or more non-radiopaque markers, for example, formed by laser etching, on one or more portions of shaft 14 (e.g., on an outer diameter of outer coating 70).

Various aspects of this disclosure may help to improve the column strength of shaft 14 or electrode 26, or otherwise improve the usability of shaft 14 and/or electrode 26. For example, various aspects of this disclosure (i.e., braiding 60 with threads 62) may help to allow for shaft 14 and/or electrode 26 to be delivered to a treatment site, for example, through, deflected by, and/or otherwise manipulated with, an insertion device. Additionally, various aspects of this disclosure (e.g., braiding 60 with threads 62) may help to allow for shaft 14 and/or electrode 26 to be extended and/or retracted (e.g., actuated) relative to an insertion device. Similarly, various aspects of this disclosure (e.g., braiding 60 with threads 62) may help to allow for electrode 26 to be extended and/or retracted (e.g., actuated) relative to shaft 14.

In some aspects, various aspects of this disclosure (e.g., braiding 60 with threads 62) may help to allow for shaft 14 and/or electrode 26 to be extended and/or retracted (e.g., actuated) relative to the insertion device when a distal portion of the insertion device is in a retroflex position or orientation. For example, various aspects of this disclosure (e.g., braiding 60 with threads 62) may help to allow for a user to more accurately and/or predictably extend, retract, or otherwise actuate shaft 14 and/or electrode 26, for example, by extending or retracting medical device 10 and/or extending or retracting movable body 20. In these aspects, braiding 60 with threads 62 may help to allow for a consistent correlation (e.g., a one-to-one, real-time, non-delayed relationship) between the extension and/or retraction of handle 12 (e.g., relative to a proximal end of an insertion device, not shown) and the extension and/or retraction of shaft 14 and electrode 26 (e.g., relative to the distal end of the insertion device and/or the treatment site). Moreover, in these aspects, braiding 60 with threads 62 may help to allow for a consistent correlation (e.g., a one-to-one, real-time, non-delayed relationship) between the extension and/or retraction of movable body 20 (e.g., relative to main body 18) and the extension and/or retraction of electrode 26 (e.g., relative to end cap 50 and/or shaft 14).

Moreover, various aspects of this disclosure may help to allow for shaft 14 and/or electrode 26 to be more flexible and/or stronger, for example, for use in an Endoscopic Mucosal Resection (EMR), Endoscopic Sub-mucosal Resection (ESR), Endoscopic Sub-mucosal Dissection (ESD), polypectomy, mucosectomy procedures, or in peroral endoscopic myotomy (POEM) procedures. In these aspects, braiding 60 with threads 62 may help to prevent and/or minimize shaft 14, or the drive element connecting movable body 20 and electrode 26, from kinking or otherwise deforming in such a way that extension or retraction of shaft 14 and/or electrode 26 may be impaired. In some aspects, braiding 60 with threads 62 may help to prevent or minimize axial elongation and/or help to maximize lumen patency (e.g., as shafts tend to “fish eye” around an arch or bend). Furthermore, outer coating 70 may help to insulate or otherwise encapsulate braiding 60 and threads 62 (or other portions or components of shaft 14), for example, helping to protect tissue at the treatment site and/or helping to protect the user. In some aspects, outer coating 70, for example, formed of PEEK, may help to bias or create partial shape memory properties for shaft 14. Furthermore, if threads 62 of braiding 60 are formed of a plastic or non-metallic material (e.g., a polymeric material), threads 62 may further help to protect tissue at the treatment site and/or help to protect the user, for example, by helping to prevent energy transfer from electrode 26 to shaft 14, the treatment site, and/or to the user.

While principles of this disclosure are described herein with reference to illustrative aspects for particular applications, it should be understood that the disclosure is not limited thereto. Those having ordinary skill in the art and access to the teachings provided herein will recognize additional modifications, applications, aspects, and substitution of equivalents all fall within the scope of the aspects described herein. Accordingly, the disclosure is not to be considered as limited by the foregoing description.

We claim:

1. A medical device, comprising:
a handle, including a movable body;
a shaft extending from the handle; and
an electrode at a distal end of the shaft,
wherein movement of the movable body of the handle controls a position of the electrode relative to the distal end of the shaft, and
wherein at least a portion of the shaft includes a braiding.

2. The medical device of claim 1, wherein the braiding is a polymer, and wherein the medical device is an electrosurgical knife.

3. The medical device of claim 1, wherein the shaft further includes an outer coating, and wherein the braiding is embedded within the outer coating.

4. The medical device of claim 3, wherein the braiding includes a plurality of threads over at least a portion of the shaft.

5. The medical device of claim 4, further comprising an inner shaft, and wherein the plurality of threads are deposited over at least a portion of the inner shaft.

6. The medical device of claim 5, wherein the braiding extends along an entire length of the inner shaft.

7. The medical device of claim 6, wherein the outer coating is formed of polyether ether ketone (PEEK).

8. The medical device of claim 1, wherein the handle includes a port and an electrical connection, wherein the handle and the shaft connect the port and the electrical connection to the electrode.

9. The medical device of claim 1, wherein movement of the movable body of the handle controls a position of the electrode relative to the distal end of the shaft without delay.

10. The medical device of claim 1, wherein the braiding is configured to minimize kinking of the shaft or a drive element connecting the movable body to the electrode.

11. The medical device of claim 1, wherein a distal portion of the shaft further includes a ring radially surrounding a portion of the braiding.

12. The medical device of claim 11, wherein the ring is a radiopaque marker band.

13. The medical device of claim 12, further comprising:
an end cap coupled to the distal end of the shaft, wherein a portion of the end cap extends distally of the distal end of the shaft to form an exposed portion of the end cap, wherein the end cap is at least partially insulative, and wherein the ring is positioned proximal of the exposed portion of the end cap.

14. The medical device of claim 1, wherein the handle includes one or more indicators, wherein the one or more indicators are indicative of at one or more configurations of the electrode relative to the distal end of the shaft.

15. The medical device of claim 14, wherein the one or more indicators includes a first indicator and a second indicator, wherein the first indicator is indicative of a retracted configuration of the electrode, and wherein the second indicator is indicative of an extended configuration of the electrode.

16. A medical device, comprising:
a handle;
a shaft extending from the handle; and
an electrosurgical knife at a distal end of the shaft,
wherein at least a portion of the shaft includes a braiding that includes a plurality of polymer threads.

17. The medical device of claim 16, wherein the handle includes a movable body, and wherein movement of the movable body controls a position of the electrosurgical knife relative to the distal end of the shaft.

18. The medical device of claim 17, including an inner shaft, wherein the plurality of polymer threads are deposited over at least a portion of the inner shaft, wherein the shaft further includes an outer coating formed of polyether ether ketone (PEEK), and wherein the braiding is embedded within the outer coating.

19. A medical device, comprising:
a handle, including a movable body;
a shaft extending from the handle; and
an electrode at a distal end of the shaft,
wherein movement of the movable body of the handle controls a position of the electrode relative to the distal end of the shaft,
wherein at least a portion of the shaft includes a braiding that includes a plurality of threads over at least a portion of the shaft.

20. The medical device of claim 19, further comprising an inner shaft, wherein the plurality of threads are deposited over at least a portion of the inner shaft,
wherein the shaft further includes an outer coating, and
wherein the braiding is embedded within the outer coating.
